(19) United States
(12) Patent Application Publication
(10) Pub. No.: US 2024/0350409 A1
(43) Pub. Date: Oct. 24, 2024

(54) TRANSDERMAL DELIVERY FORMULATIONS AND METHODS FOR THE MANUFACTURE THEREOF

(71) Applicant: LifeActive, Inc., Grants Pass, OR (US)

(72) Inventor: Calvin Dexter Morris, Medford, OR (US)

(73) Assignee: LifeActive, Inc., Grants Pass, OR (US)

(21) Appl. No.: 18/142,619

(22) Filed: May 3, 2023

Related U.S. Application Data
(60) Provisional application No. 63/460,812, filed on Apr. 20, 2023.

(51) Int. Cl. A61K 9/107; A61K 9/00; A61K 45/06; A61K 47/12; A61K 47/26; A61K 47/44

(52) U.S. Cl. CPC A61K 9/1075; A61K 9/0014; A61K 45/06; A61K 47/12; A61K 47/26; A61K 47/44

(57) ABSTRACT

Provided are transdermal delivery formulations, and methods for the manufacture thereof, for the epicutaneous administration of drugs and nutrients to a human subject or domestic, veterinary, or agricultural animal. Transdermal delivery formulations disclosed herein comprise a homogenous mixture of (a) a transdermal accelerant comprising a weak organic acid having a pKa greater than 2.0 and (b) a microemulsion comprising a nonionic emulsifier, water, and a cis-unsaturated long-chain fatty acid. Fatty acid microemulsions are combined with the acidified transdermal accelerants having a pH greater than 1.0 (typically from 1.5 to 2.5) to yield a homogenous transdermal delivery formulation comprising fatty acid micelles and/or liposomes that encapsulate one or more compound, such as a nutrient or a drug, and incorporate one or more cis-unsaturated fatty acid having a 12-26 carbon chain that includes one or more double bond in a cis configuration.

### CROSS-REFERENCE TO RELATED APPLICATIONS

This U.S. non-provisional patent application was filed on May 3, 2023 as U.S. patent application Ser. No. 18/142,619 and claims the benefit of U.S. Provisional Patent Application No. 63/460,812, which was filed on Apr. 20, 2023. The contents of U.S. Provisional Patent Application No. 63/460,812 are incorporated herein by reference in their entirety.

### BACKGROUND OF THE DISCLOSURE

### Technical Field

The present disclosure relates, generally, to the fields of nutrition and medicine, in particular to transdermal delivery formulations for the epicutaneous administration of compounds, in particular drugs and nutrients, and methods for the manufacture of transdermal delivery formulations. The transdermal delivery formulations disclosed herein comprise a homogenous mixture of (a) a transdermal accelerant comprising a weak organic acid and (b) a microemulsion comprising a nonionic emulsifier, water, and a cis-unsaturated long-chain fatty acid.

### Description of the Related Art

Technologies and methods for achieving the systemic in vivo delivery of drugs and nutrients to a human subject or domestic, veterinary, or agricultural animal are generally categorized on the basis of whether the drugs and/or nutrients are administered enterally (i.e. via the gastrointestinal “GI” tract) or parenterally (i.e. avoiding the intestines). Reviewed in Finkel, Lippincott's Illustrated Reviews: Pharmacology (8^{th} Ed., 2022) ISBN 978-1605472003 and Humphrey, Rang and Dale's Pharmacology (7^{th} Ed., 2007) ISBN 978-0702034718.

In contrast to enteral administration, drugs and nutrients administered parenterally avoid the GI tract altogether and enter the bloodstream via routes other than intestinal absorption. Because transdermal delivery bypasses the gastrointestinal tract, drugs and nutrients are not subject to the enzymatic and pH associated deactivation that is commonly associated with enteral delivery. Moreover, transdermal delivery avoids hepatic first-pass metabolism, minimized drug interactions with ingested food or other orally administered drugs, and is associated with improved patient compliance, permits controlled drug and nutrient delivery with minimal side effects and improved bioavailability, and is suitable for use with nauseated patients. Kadam, IJRDPLS 3(4):1042 (2014) and Finnin, J. Pharm. Sci. 88(10):955 (1999).

Parenteral delivery of drugs and nutrients through the skin can be accomplished by either (1) topical administration to achieve localized delivery and therapeutic effect or (2) transdermal administration via epicutaneous delivery of a drug and/or nutrient to the bloodstream to achieve systemic delivery and therapeutic effect. Transdermal delivery technologies available to date for epicutaneous delivery of a drug and/or nutrient predominantly employ a patch and have limited utility for the delivery of high doses of drugs and nutrients.

Nonetheless, transdermal drug delivery has made an important contribution to medical practice. First-generation transdermal delivery systems have steadily increased in clinical use in the delivery of small, lipophilic, low-dose drugs. Second-generation delivery systems using chemical enhancers, non-cavitational ultrasound, and iontophoresis have also resulted in clinical products; the ability of iontophoresis to control delivery rates in real time provides added functionality. Third-generation delivery systems target their effects to the skin's barrier layer of stratum corneum by using microneedles, thermal ablation, microdermabrasion, electroporation, and cavitational ultrasound. Microneedles and thermal ablation are currently progressing through clinical trials for delivery of macromolecules and vaccines, such as insulin, parathyroid hormone, and influenza vaccine. Prausnitz, Nature Biotech. 26(11):1261 (2008).

U.S. Pat. No. 8,784,878 (“Morgan”) discloses non-occlusive transdermal pharmaceutical compositions for the delivery of testosterone, comprising the penetration enhancers octyl salicylate and Padimate O and a volatile solvent selected from ethanol and isopropanol.

U.S. Pat. No. 9,078,810 and PCT Patent Publication No. WO 2009/055859 (“Setiawan”) disclose transdermal delivery systems comprising a composition comprising a physiologically active agent and a penetration enhancer comprising a combination of (i) an ester of salicylic acid and (ii) polyethylene glycol (PEG) of average molecular weight no more than 300.

U.S. Pat. No. 9,180,194 (“Dipietro”) discloses a transdermal delivery composition comprising a physiologically active agent, a volatile solvent, and a viscosity modulating agent.

U.S. Pat. No. 9,867,881 (“Soane”) discloses formulations for the delivery of concentrated protein solutions comprising a lower viscosity liquid formulation or a higher concentration of therapeutic or nontherapeutic proteins as compared to traditional protein solutions.

U.S. Pat. No. 10,744,078 (“Dake”) discloses formulations for transdermal delivery of botulinum toxin, which formulations comprise a partitioning agent, an oligo- or polyanion-bridge, and, optionally, a viscosity modifying agent.

U.S. Pat. No. 10,987,316 (“Liao”) discloses transdermal drug delivery patches for the transdermal administration of tertiary amine drugs, such as rivastigmine, fentanyl, or rotigotine, which systems comprise a polymer matrix comprising a free base form of a drug and at least one carboxyl group-containing compound.

U.S. Pat. No. 11,129,975 (“Ross”) discloses a device for delivering a high viscosity composition, comprising microneedles with structures fabricated on a surface to form a microtopography.

U.S. Patent Publication No. 2017/0232115 (“Ashley”) discloses microparticle protocells comprising a microporous silica core with a supported lipid bilayer for targeting of hepatocellular and other cancer cells.

U.S. Patent Publication No. 2018/0110739 (“Yum”) discloses transdermal delivery systems comprising bupivacaine.

U.S. Patent Publication No. 2020/0023173 (“Smith”) discloses methods for delivering permeant substances transdermally into a membrane of an animal via an opening in the skin tissue.

PCT Patent Publication No. WO 2009/158032 (“Berenson”) discloses patches and methods for the transdermal delivery of therapeutically effective amounts of iron.

PCT Patent Publication No. WO 2009/158120 (“Tang”) discloses a solid dispersion transdermal drug delivery system comprising a therapeutic agent in a stable amorphous form and a combination polymeric stabilizing and dispersing agent having a hydrogen bond-forming functional group.

The non-invasive transdermal delivery of compounds, including drugs and nutrients, has the potential for providing enhanced bioavailability, improved solubility, bypass of the first-pass metabolism, and targeted delivery of drugs in brain-related disorders. Yet, despite the availability of technologies for the transdermal administration of some drugs and nutrients, transdermal delivery formulations that are currently available in the art fail to achieve substantial penetration of both the stratum corneum and the epidermis and, thus, are unsuitable for the systemic delivery of compounds. Thus, there remains an unmet need in the art for transdermal formulations that achieve the epicutaneous administration of compounds into the bloodstream of a human subject or domestic, veterinary, or agricultural animal.

### SUMMARY OF THE DISCLOSURE

The present disclosure fulfills unmet needs in the art for efficient and systemic epicutaneous administration of drugs, nutrients, and other compounds to a human subject or domestic, veterinary, or agricultural animal. The transdermal delivery formulations disclosed herein provide unexpected and surprising advantages over existing technologies for the transdermal delivery of drugs and nutrients to achieve the efficient and systemic epicutaneous administration of compounds, including drugs and nutrients, to a human subject or domestic, veterinary, or agricultural animal in need thereof.

Transdermal delivery formulations disclosed herein comprise acidified micelles and/or liposomes that encapsulate a compound, such as a nutrient or a drug. These transdermal delivery formulations are made by preparing separately (a) a transdermal accelerant comprising a weak organic acid solution having a pKa greater than 2.0 and (b) a microemulsion comprising a nonionic emulsifier, water, and a cis-unsaturated long-chain fatty acid. Those fatty acid microemulsions are combined with the acidified transdermal accelerants having a pH greater than 1.0 (typically from 1.5 to 2.5) to yield a homogenous transdermal delivery formulation comprising fatty acid micelles and/or liposomes that encapsulate one or more compound, such as a nutrient or a drug. Those micelles and/or liposomes incorporate one or more cis-unsaturated fatty acid having a 12-26 carbon chain that includes one or more double bond in a cis configuration. The resulting micelles and/or liposomes yield transdermal delivery formulations that exhibit ideal solubility and absorption properties in high humidity conditions, such as in a warm to hot shower or sauna.

In some embodiments, the transdermal delivery formulations of the present disclosure comprise a homogenous mixture of (a) a transdermal accelerant comprising a weak organic acid, typically having a pKa greater than 2.0, and (b) a microemulsion comprising a nonionic emulsifier, water (in particular distilled water) and an unsaturated long-chain fatty comprising a chain of from 12 to 26 carbons (in particular, having from 1 to 2 double bonds in a cis configuration).

Within certain aspects of these embodiments, transdermal delivery formulations employ a transdermal accelerant comprising a weak organic acid having a median pKa of from 2.0 to 6.0, or a median pKa of from 3.0 to 5.5, or a median pKa of from 4.0 to 5.0, or a median pKa of about 4.7. Within related aspects, the weak organic acid is selected from the group consisting of lactic acid, acetic acid, formic acid, citric acid, oxalic acid, uric acid, malic acid, maleic acid, tartaric acid, malonic acid, succinic acid, and fumaric acid. Exemplified herein are transdermal delivery formulations wherein the weak organic acid is citric acid, acetic acid, or a combination of citric acid and acetic acid.

Within other aspects of these embodiments, transdermal delivery formulations comprise a microemulsion, such as a cream, an ointment, a liniment, a paste, a film, or a liquid, which comprises a nonionic emulsifier that is selected from the group consisting of lecithin, carboxylmethylcellulose, a sorbitan ester, and a polysorbate, wherein the sorbitan ester is selected from the group consisting of sorbitan monolaurate, sorbitan monostearate, sorbitan tristearate, and sorbitan monooleate or wherein the polysorbate is selected from the group consisting of polyoxyethylene (20) sorbitan monolaurate (Polysorbate 20), polyoxyethylene (20) sorbitan monopalmitate (Polysorbate 40), polyoxyethylene (20) sorbitan monostearate (Polysorbate 60), and polyoxyethylene (20) sorbitan monooleate (Polysorbate 80). Exemplified herein are transdermal delivery formulations wherein the microemulsion comprises the nonionic emulsifier Polysorbate 80.

Within related aspects of these embodiments, transdermal delivery formulations comprise a microemulsion comprising one or more unsaturated long-chain fatty acid having a chain of from 12 to 26 carbons and having one or more double bond in a cis configuration. Preferred are cis-unsaturated long-chain fatty acids having one or more cis double bond at position 3-10, or at position 4-8, or at position 5-7.

Representative long-chain fatty acids may be selected from the group consisting of Sapienic Acid, Palmitoleic Acid, Margoleic Acid, Cis-Vaccenic Acid, Oleic Acid, Petroselinic Acid, Linoleic Acid, Eicosenoic Acid, Gadoleic Acid, Eicosadienoic Acid, Erucic Acid, Docosadienoic Acid, and Nervonic Acid. Exemplified herein are transdermal delivery formulations wherein the 16 to 26 carbon unsaturated long-chain fatty acid is Oleic Acid, Linoleic Acid, or a combination of Oleic Acid and Linoleic Acid.

Within further related aspects of these embodiments, transdermal delivery formulations comprise a microemulsion wherein one or more unsaturated long-chain fatty acid having a chain of from 16 to 26 carbons and having one or more double bond in a cis configuration is comprised within a plant oil, including a vegetable oil, a nut oil, and/or a seed oil. Representative plant oils may be selected from the group consisting of Macadamia Oil, Maracuja (Passion Fruit) Oil, Safflower Oil, Sunflower Oil, Olive Oil, Avacado Oil, Canola Oil, Coconut Oil, Corn Oil, Cottonseed Oil, Flaxseed/Linseed Oil, Grape Seed Oil, Hemp Seed Oil, Palm Oil, Peanut Oil, Rice Bran Oil, Sesame Oil, Soybean Oil, Brazil Nut Oil, Almond Oil, Walnut Oil, and Pecan Oil. Exemplified herein are transdermal delivery formulations wherein the plant oil is selected from the group consisting of Macadamia Oil, Maracuja (Passion Fruit) Oil, or a combination of Macadamia Oil and Maracuja (Passion Fruit) Oil.

In other embodiments, transdermal delivery formulations further comprise a nitrate source, such as a plant-based nitrate source including, for example, a plant-based nitrate source is selected from the group consisting of arugula, spinach, and beetroot. Exemplified herein are transdermal delivery formulations wherein the plant-based nitrate source is beetroot.

In yet other embodiments, transdermal delivery formulations further comprise one or more (a) viscosity enhancer, (b) nutrient, (c) plant powder or extract, (d) amino acid, and/or (e) vitamin. Representative viscosity enhancers may be selected from the group consisting of lecithin, aloe vera, glycerin, plant oil, animal oil, and collagen. Representative nutrients may be selected from the group consisting of acetyl-L-carnitine, alpha lipoic acid, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide. Representative plant powders or extracts may be selected from the group consisting of bacopa powder, bamboo extract powder, beet powder, blueberry extract, ginko biloba, ginger, grape seedextract, green tea, jojoba, nutmeg, olive leaf, pomegranate, and turmeric. Representative amino acids may be selected from the group consisting of alanine, arginine, leucine, isoleucine, valine, glutamine, glycine, histidine, leucine, lysine, methionine, proline, serine, threonine, and valine. Representative vitamins may be selected from the group consisting of vitamin A, vitamin B, vitamin B-3, vitamin B-7 & 8 inositol, vitamin B-9 (folic acid), vitamin B-12, vitamin C, vitamin D-3, and vitamin E.

The presently disclosed transdermal delivery formulations typically have (1) a pH of from 1.0 to 6.0 or from 2.0 to 5.0 or from 3.0 to 5.0 and (2) a viscosity at 20° C. of from 500 to 10,000 centipoise (cP) or from 1,000 to 5,000 cP, or from 1,500 to 4,000 cP, or from 2,000 to 3,000 cP, or about 2,500 cP.

The presently disclosed transdermal delivery formulations typically employ fatty acid microemulsions comprising equal parts nonionic surfactant and unsaturated fatty acid on a volume to volume or mass to mass basis.

These and other related aspects of the present disclosure will be better understood in view of the following drawings and detailed description, which exemplify certain aspects of the various embodiments.

### BRIEF DESCRIPTION OF THE DRAWINGS

Certain aspects of the present disclosure will become more evident in reference to the drawings, which are presented for illustration, not limitation.

FIG. 1 is a drawing that depicts the major elements of human skin, including, in sequence from outer layer to inner layer, the epidermis, the dermis, and the hypodermis. As shown, the epidermis comprises an outer surface layer of stratum corneum, which covers the stratum lucidum, the stratum granulosum, stratum spinosum, and basal layer at the inner surface of the epidermis. Vascularization originates at the interface between the dermis and hypodermus, and capillaries extend into the dermis.

FIG. 2 is a drawing that depicts liposomes and micelles comprised within cis-unsaturated fatty acid microemulsions and transdermal delivery formulations disclosed herein.

FIG. 3 is a drawing that depicts a Franz diffusion cell for use in in vitro models for testing transdermal delivery formulations as disclosed in Example 1 by, for example, utilizing non-viable skin to measure penetration and permeation only or utilizing fresh, metabolically active skin to simultaneously measure permeation and skin metabolism. From, Bartosova, Current Medicinal Chemistry 19:4671 (2012).

FIG. 4 provides bar graphs (FIGS. 4A-4F) depicting the data presented in Example 3, Table 7.

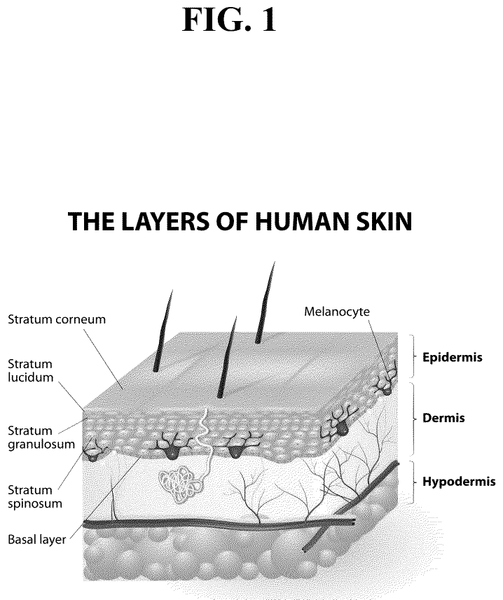

### DETAILED DESCRIPTION OF THE DISCLOSURE

Provided herein are transdermal delivery formulations that exhibit unexpected and surprising advantages over technologies that are currently available in the art for the efficient systemic epicutaneous administration of drugs, nutrients, and other compounds to the bloodstream of a human subject or domestic, veterinary, or agricultural animal. Transdermal delivery formulations disclosed herein comprise a homogenous mixture of (a) a transdermal accelerant comprising a weak organic acid having a pKa greater than 2.0 and (b) a microemulsion comprising a nonionic emulsifier, distilled water, and a cis-unsaturated long-chain fatty acid.

Transdermal delivery formulations disclosed herein comprise acidified micelles and/or liposomes that encapsulate a compound, such as a nutrient or a drug. These transdermal delivery formulations are made by preparing separately (a) a transdermal accelerant comprising a weak organic acid solution having a pKa greater than 2.0 and (b) a microemulsion comprising a nonionic emulsifier, water, and a cis-unsaturated long-chain fatty acid. Those fatty acid microemulsions are combined with the acidified transdermal accelerants having a pH greater than 1.0 (typically from 1.5 to 2.5) to yield a homogenous transdermal delivery formulation comprising fatty acid micelles and/or liposomes that encapsulate one or more compound, such as a nutrient or a drug. Those micelles and/or liposomes incorporate one or more cis-unsaturated fatty acid having a 12-26 carbon chain that includes one or more double bond in a cis configuration. The resulting micelles and/or liposomes yield transdermal delivery formulations that exhibit ideal solubility and absorption properties in high humidity conditions, such as in a warm to hot shower or sauna.

This disclosure will be better understood in view of the following definitions, which are provided for clarification and are not intended to limit the scope of the subject matter that is disclosed herein.

### Definitions

The practice of the present disclosure will employ conventional technologies and methods that are in common use in the fields of nutrition and medicine, in particular in conjunction with epicutaneous administration of drugs, nutrients, and other compounds. Such technologies and methods are explained fully in treatises on drug and nutritional delivery as well as medical, scientific, and patent literature. See, e.g., Franz, “Transdermal Delivery” and Kydonieus, “Fundamentals-Optimization, and Applications,” both in Treatise on Controlled Delivery (CRC Press, 2017); Florence, “Physicochemical Principles of Pharmacy in Manufacture, Formulation, and Clinical Use” PhP (London, 2016); and Benson, “Topical and Transdermal Drug Delivery: From Simple Potions to Smart Technologies” Current Drug Delivery 16 (5): 440-460 (2019). Unless specifically defined otherwise herein, each term used in this disclosure has the same meaning as it would to those having skill in the relevant art.

As used herein, the terms “drug delivery/administration” and “nutrient delivery/administration” refer, generally, to the enteral and parenteral delivery of drugs and/or nutrients to a mammal, in particular a human, subject. “Enteral drug and nutrient delivery/administration” involves passage through the gastrointestinal tract and includes oral, sublingual, and rectal delivery. While oral delivery is the most common route for the enteral administration of drugs and nutrients, it is unsuitable for delivery of drugs and nutrients that are unstable in the acidic environment of the stomach, which can destroy their biological activity or otherwise block bioavailability. Many drugs and nutrients are poorly absorbed in the intestines and/or are subject to first-pass metabolism wherein the concentration of an active drug or nutrient enters the hepatic portal system and is absorbed and/or metabolized in the liver before reaching the site of action or systemic circulation. Rowland, J. Pharm. Sci. 61(1):70-74 (1972) and Pond, Clin. Pharm. 9(1):1-25 (1984). Drugs or nutrients administered sublingually (i.e. placed under the tongue) diffuse into the capillary network, are rapidly absorbed, and enter systemic circulation directly, thereby avoiding the gastrointestinal tract and are less susceptible to first-pass metabolism. Moreover, oral delivery is impractical for delivery to unconscious patients or when acute onset is required.

As used herein, the terms “parenteral delivery” and “parenteral administration” refer synonymously to the in vivo administration (to a human subject or domestic, veterinary, or agricultural animal) of compounds, including drugs and nutrients, via routes that avoid the gastrointestinal tract altogether and that enter the bloodstream via routes other than intestinal absorption. Parenteral administration of drugs and nutrients includes both topical (local) administration and systemic administration via delivery to the bloodstream.

As used herein, the terms “transdermal delivery/administration” and “epicutaneous delivery/administration” refer interchangeably to the non-enteral, parenteral delivery/administration of drugs, nutrients, and other compounds to a subject through the skin to, thereby, avoid the adverse, degradative environment of the stomach and inefficient delivery of biologically active molecules through the small and large intestines in favor of the direct, non-invasive, and efficient delivery of molecules through the skin.

As used herein, the terms “transdermal delivery” and “transdermal penetration” refer synonymously to the passive diffusion of drugs, nutrients, and other compounds from the outer surface of the skin, through the stratum corneum and epidermis, and into the blood vasculature or via a shunt pathway, such as through hair follicles and associated sebaceous glands and the sweat ducts. Barry, J. Controlled Release 6(1):85 (1987); Bodde, J. Controlled Release 15(3):227 (1991); and Heisig, Pharm. Res. 13:421 (1996).

As used herein, the term “skin” refers primarily to “human skin,” which comprises three distinct but mutually dependent tissues, namely: 1. The stratified, a vascular, cellular epidermis; 2. Underlying dermis of connective tissues; and; 3. Hypodermis. Tortora, “Principles of Anatomy & Physiology” (XI Ed., John Wiley & Sons, 2006) and Wilson, “Anatomy and Physiology in Health and Illness (1996).

As used herein, the term “stratum corneum” refers to the outermost layer of skin. The stratum corneum is approximately 10 mm thick when dry but swells to several times this thickness when fully hydrated. It contains 10 to 25 layers parallel to the skin surface, which include dying or dead, keratinized cells, called corneocytes. Stratum corneum is flexible but relatively impermeable. The stratum corneum is the principal barrier for penetration. The barrier nature of the stratum corneum depends critically on its constituents: 75 to 80% proteins, 5 to 15% lipids, and 5 to 10% ondansetron material on a dry weight basis. Protein fractions predominantly contain alpha-keratin (70%) with some beta-keratin (10%) and cell envelope (5%). Lipid constituents vary with body site (neutral lipids, sphingolipids, polar lipids, cholesterol). Phospholipids are largely absent, a unique feature of mammalian membrane.

As used herein, the term “epidermis” refers to the multilayered envelop of the epidermis varies in thickness, depending on cell size and number of cell layers, ranging from 0.8 mm on palms and soles down to 0.06 mm on the eyelids. Stratum corneum and the remainder of the epidermis, also called viable epidermis, cover a major area of skin.

As used herein, the term “viable epidermis” refers to the cell layer that is situated beneath the stratum corneum, which varies in thickness from 0.06 mm on the eyelids to 0.8 mm on the palms. Going inwards, it includes various layers as stratum lucidum, stratum granulosum, stratum spinosum, and the stratum basale. In the basale layer, mitosis of the cells constantly renews the epidermis and this proliferation compensates the loss of dead horney cells from the skin surface. As the cells produced by the basale layer move outward, they alter morphologically and histochemically, undergoing keratinization to form the outermost layer of stratum corneum.

As used herein, the term “dermis” refers to the 3 to 5 mm thick layer and is composed of a matrix of connective tissue which contains blood vessels, lymph vessels, and nerves. The continuous blood supply has essential function in regulation of body temperature. It also provides nutrients and oxygen to the skin while removing toxins and waste products. Capillaries reach to within 0.2 mm of skin surface and provide sink conditions for most molecules penetrating the skin barrier. The blood supply thus keeps the dermal concentration of permeate very low, and the resulting concentration difference across the epidermis provides the essential driving force for transdermal permeation.

As used herein, the term “hypodermis” refers to the subcutaneous fat tissue that supports the dermis and epidermis. The hypodermis serves as a fat storage area, which helps to regulate temperature and provides nutritional support and mechanical protection. “Hypodermis” carries principal blood vessels and nerves to skin and may contain sensory pressure organs. For transdermal drug delivery, the drug has to penetrate through all these three layers and reach into systemic circulation while in case of topical drug delivery, only penetration through stratum corneum is essential and then retention of drug in skin layers is desired.

As used herein, the terms “transcorneal delivery” and “transcorneal penetration” refer synonymously to both the “intracellular” and “intercellular” penetration of a compound past the stratum corneum.

“Intracellular transcorneal delivery” and “intracellular transcorneal penetration” refer to the passing of a compound, typically a “hydrophilic compound” through the cells of the stratum corneum. As stratum corneum hydrates, water accumulates near the outer surface of the protein filaments. Polar molecules appear to pass through this immobilized water.

“Intercellular transcorneal delivery” and “intercellular transcorneal penetration” refer to the passing of a compound, typically a “hydrophobic compound” through the cells of the stratum corneum by dissolve in and diffuse through the non-aqueous lipid matrix imbibed between the protein filaments.

As used herein, the terms “transappendegeal delivery” and “transappendegeal penetration” refer synonymously to the shunt pathway whereby a compound traverses through the hair follicles, the sebaceous pathway of the pilosebaceous apparatus, and/or the aqueous pathway of the salty sweat glands. The transappendegeal pathway is considered to be of minor importance because of its relatively smaller area (less than 0.1% of total surface). This route is of substantial relevance for the “transappendegeal delivery” or “transappendegeal penetration” polar, hydrophobic, and/or lipophilic compounds.

As used herein, the term “weak organic acid” refers to a compound that partially dissociates when dissolved in a solvent, in particular, a fatty acid microemulsion. The strength of a weak acid can be quantified in terms of a dissociation constant, defined as “pH,” which refers to the negative logarithm of the H ion concentration.

As used herein, the term “pKa” refers to acid dissociation constant (Ka) of a solution. The pH of a solution can be predicted when the analytical concentration and pKa values of all acids and bases are known; conversely, it is possible to calculate the equilibrium concentration of the acids and bases in solution when the pH is known. These calculations find application in many different areas of chemistry, biology, medicine, and geology. For example, many compounds used for medication are weak acids or bases, and a knowledge of the pKa values. The quantitative behavior of acids and bases in solution can be understood only if their pKa values are known. pKa describes the acidity of a particular molecule. It measures the strength of an acid by how tightly a proton is held by a Bronsted acid. The lower the value of pKa, the stronger the acid and the greater its ability to donate its protons. describe the acidity of a particular molecule Ka denotes the acid dissociation constant. It measures how completely an acid dissociates in an aqueous solution. The larger the value of Ka, the stronger the acid as acid largely dissociates into its ions and has lower pKa value.

The relationship between pKa and Ka is described by the following equation:

$$pKa = - log [ Ka ]$$

Acid dissociation constants, or pKa values, are essential for understanding many fundamental reactions in chemistry. These values reveal the deprotonation state of a molecule in a particular solvent. There is great interest in using theoretical methods to calculate the pKa values for many different types of molecules.

As used herein, the term “strong acids” refers to those acids that exhibit a negative pKa are that are unsuitable for use in the transdermal delivery formulations disclosed herein. Exemplary “strong acids” are presented in Table 1.

TABLE 1
Acidic Dissociation Constants (pKa) of Strong Acids

| | | |
|---|---|---|
| | | pKa |
| | | (in H₂O at |
| Strong Acids | Structure | ~15-20° C.) |

| | | | |
|---|---|---|---|
| Hydrochloric Acid | HCl | pKa | −5.9 |
| Hydrobromic Acid | HBr | pka | −8.8 |
| Hydroiodic Acid | HI | pKa | −9.5 |
| Triflic Acid | H[CF₃SO₃] | pKa | −14 |
| Perchloric Acid | H[ClO₄] | pKa | −15 |
| Nitric Acid | HNO₃ | pKa | −1.6 |
| Sulfuric Acid | H₂SO₄ | pKal | −3 |

As used herein, the term “unsaturated fatty acid” refers to a fatty acid comprising one or more C═C double bonds. C═C double bonds can adopt either a “cis” or a “trans” configuration and thereby yield either a “cis unsaturated fatty acid” or a “trans unsaturated fatty acid.”

As used herein, the term “cis unsaturated fatty acid” refers to a fatty acid comprising one or more C═C double bonds in a “cis” configuration, wherein two hydrogen atoms adjacent to the double bond stick out on the same side of the C═C chain. The rigidity of a double bond freezes its conformation and, in the case of the cis isomer, causes the chain to bend and restricts the conformational freedom of the fatty acid. The more double bonds the chain has in the cis configuration, the less flexibility it has. When a chain has many cis bonds, it becomes quite curved in its most accessible conformations.

For example, oleic acid, with one double bond, has a “kink” in it, whereas linoleic acid, with two double bonds, has a more pronounced “bend.” α-Linolenic acid, with three double bonds, favors a “hooked” shape. The effect of this is that, in restricted environments, such as when fatty acids are part of a phospholipid in a lipid bilayer or triglycerides in lipid droplets, “cis” bonds limit the ability of fatty acids to be closely packed, and therefore can reduce the melting temperature of the membrane or of the fat and, thereby, “cis unsaturated fatty acids” increase cellular membrane fluidity as compared to the “trans unsaturated fatty acid” comprising the same atomic constituents/primary molecular structure.

As used herein, the term “trans unsaturated fatty acid” refers to a fatty acid comprising one or more C═C double bonds in a “trans” configuration, wherein two hydrogen atoms adjacent to the double bond lie on opposite sides of the chain. As a result, they do not cause the chain to bend much, and their shape is similar to straight saturated fatty acids. In most naturally occurring unsaturated fatty acids, each double bond has three (n-3), six (n-6), or nine (n-9) carbon atoms after it, and all double bonds have a cis configuration.

Words and phrases using the singular or plural number also include the plural and singular number, respectively. For example, terms such as “a” or “an” and phrases such as “at least one” and “one or more” include both the singular and the plural. Terms that are intended to be “open” (including, for example, the words “comprise,” “comprising,” “include,” “including,” “have,” and “having,” and the like) are to be construed in an inclusive sense as opposed to an exclusive or exhaustive sense. That is, the term “including” should be interpreted as “including but not limited to,” the term “includes” should be interpreted as “includes but is not limited to,” the term “having” should be interpreted as “having at least.”

The use of the term “or” in the claims is used to mean “and/or” unless explicitly indicated to refer to alternatives only or the alternatives are mutually exclusive, although the disclosure supports a definition that refers to only alternatives and “and/or.”

Additionally, the terms “herein,” “above,” and “below,” and words of similar import, when used in this application, shall refer to this application as a whole and not to any particular portion of the application.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical, medical, and/or scientific publications are hereby incorporated by reference in their entirety.

### Transdermal Delivery Formulations

Transdermal delivery formulations disclosed herein comprise acidified micelles and/or liposomes that encapsulate a compound, such as a nutrient or a drug. These transdermal delivery formulations are made by preparing separately (a) a transdermal accelerant comprising a weak organic acid solution having a pKa greater than 2.0 and (b) a microemulsion comprising a nonionic emulsifier, water, and a cis-unsaturated long-chain fatty acid. Those fatty acid microemulsions are combined with the acidified transdermal accelerants having a pH greater than 1.0 (typically from 1.5 to 2.5) to yield a homogenous transdermal delivery formulation comprising fatty acid micelles and/or liposomes that encapsulate one or more compound, such as a nutrient or a drug. Those micelles and/or liposomes incorporate one or more cis-unsaturated fatty acid having a 12-26 carbon chain that includes one or more double bond in a cis configuration. The resulting micelles and/or liposomes yield transdermal delivery formulations that exhibit ideal solubility and absorption properties in high humidity conditions, such as in a warm to hot shower or sauna.

Transdermal delivery formulations according to the present disclosure exhibit unexpected and surprising advantages over technologies that are currently available in the art for the administration of drugs, nutrients, and other compounds to a human subject or domestic, veterinary, or agricultural animal—in particular, over existing technologies of the epicutaneous administration of drugs, nutrients, and other compounds. Within certain aspects, these transdermal delivery formulations comprise a homogenous mixture of (a) a transdermal accelerant comprising a weak organic acid having a pKa greater than 2.0 and (b) a microemulsion comprising a nonionic emulsifier, water, and one or more cis-unsaturated long-chain fatty acid.

The transdermal delivery formulations disclosed herein achieve the efficient and systemic transdermal delivery/permeation of a compound through the stratum corneum and epidermis and into the bloodstream of a human subject or domestic, veterinary, or agricultural animal and facilitate the rapid diffusion of compounds comprised within the transdermal delivery formulation when applied to the outer skin surface through the stratum corneum, viable epidermis, papillary dermis, and into the microcirculation.

The viable tissue layer and the capillaries are relatively permeable, and the peripheral circulation is sufficiently rapid. Hence, diffusion through the stratum corneum is traditionally the rate-limiting step. Scheuplein, “Molecular Structure and Diffusional Processes Across Intact Epidermis” (Harvard Medical School, 1967). Skin has pH of 4.2 to 5.6, solutions which have this pH range are used to avoid damage to the skin. However for a number of drugs, there may also be significant transdermal absorption at pH values at which the un-ionized form of the drug is predominant.

Once applied to the outer surface of the skin, transdermal delivery formulations traverse the skin via passive diffusion following the Fick's first law of diffusion, which relates the diffusive flux to the gradient of the concentration whereby the flux goes from regions of high concentration to regions of low concentration, at a rate and magnitude that is proportional to the concentration gradient. Thus, a compound within the transdermal delivery formulation moves from a region of high concentration (i.e., the skin surface) to a region of low concentration (i.e., the bloodstream).

Fick's first law of diffusion:

$$J = - D \frac{d φ}{dx}$$

where J is the diffusion flux, of which the dimension is the amount of substance per unit area per unit time. J measures the amount of substance that will flow through a unit area during a unit time interval. D is the diffusion coefficient or diffusivity. φ (for ideal mixtures) is the concentration, of which the dimension is the amount of substance per unit volume. x is position, the dimension of which is length. D is proportional to the squared velocity of the diffusing particles, which depends on the temperature, viscosity of the fluid, and the size of the particles according to the Stokes-Einstein relation. The driving force for the one-dimensional diffusion is the quantity −∂φ∂x, which for ideal mixtures is the concentration gradient. See, Atkins, Physical Chemistry for the Life Sciences (Oxford U. Press, 3^{rd} Ed. 2023) and Conlisk, Essentials of Micro- and Microfluidics: With Applications to the Biological and Chemical Sciences (Cambridge U. Press, 2012), which are incorporated by reference herein.

### 1. Transdermal Accelerants

Transdermal delivery formulations for epicutaneous administration of drugs, nutrients, or other compounds to a human subject or domestic, veterinary, or agricultural animal comprise a homogenous mixture of (a) a transdermal accelerant comprising a weak organic acid having a pKa greater than 2.0 and (b) a microemulsion comprising a nonionic emulsifier, water, and a cis-unsaturated long-chain fatty acid.

Transdermal accelerants according to the present disclosure are based upon the observation that weak organic acids, particularly, those having a pKa at or below the pH of the skin (presented in Table 2), when used in combination with a fatty acid microemulsion, as presented herein, greatly enhance skin permeability and delivery of drugs, nutrients, and other compounds through the skin and into the bloodstream.

TABLE 2
Median Acidic Dissociation Constants (pKa) of Weak Organic Acids

| | | | | Median |
|---|---|---|---|---|
| | | | | pKa |
| | | | | (in H₂ |
| Weak | | pKa | | at |
| Organic | | (in H₂O at | | ~15- |
| Acid | Structure | ~15-20° C.) | | 20 C.) |
| Uric Acid | 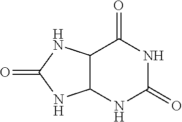 [SMILES: O=C1NC(=O)C2NC(=O)NC2N1] | pKa1 | 5.6 | 5.6 |
| Succinic Acid | 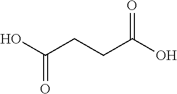 | pKa1 pKa2 | 4.2 5.6 | 4.90 |
| Acetic Acid | 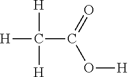 | pKa1 | 4.756 | 4.756 |
| Citric Acid | 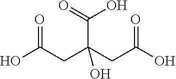 [SMILES: O=C(O)CC(O)(CC(=O)O)C(=O)O] | pKa1 pKa2 pKa3 | 3.128 4.76 6.40 | 4.76 |
| Malonic Acid | 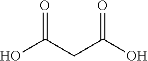 | pKa1 pKa2 pKa1 | 2.83 5.69 5.6 | 4.26 |
| Malic Acid | 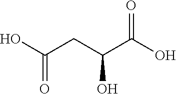 | pKa1 pKa2 | 3.40 5.20 | 3.99 |
| Maleic Acid | 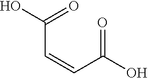 | pKa1 pKa2 | 1.90 6.07 | 3.99 |
| Lactic Acid | 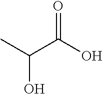 | pKa1 | 3.86 | 3.86 |
| Formic Acid | 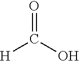 | pKa1 | 3.751 | 3.751 |
| Fumaric Acid | 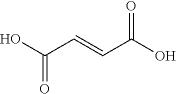 | pKa1 pKa2 | 3.03 4.44 | 3.74 |
| Tartaric Acid | 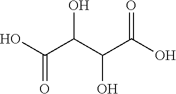 | pKa1 pKa2 | 2.89 4.40 | 3.65 |
| Oxalic Acid | 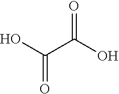 | pKa1 pKa2 pKa2 pKa2 | 1.25 4.14 5.20 6.07 | 2.7 |

Representative transdermal delivery formulations disclosed herein comprise a transdermal accelerant employing a weak organic acid solution that includes one or more weak organic acid selected from the group consisting of lactic acid, acetic acid, formic acid, citric acid, oxalic acid, uric acid, malic acid, maleic acid, tartaric acid, malonic acid, succinic acid, and fumaric acid.

Transdermal accelerants suitable for use in the manufacture of these transdermal delivery formulations comprise a weak organic acid, in particular a weak organic acid having a median pKa of from 2.0 to 6.0 or from 3.0 to 5.5 or from 4.0 to 5.0 or about 4.7, including a weak organic acid selected from the group consisting of lactic acid, acetic acid, formic acid, citric acid, oxalic acid, uric acid, malic acid, maleic acid, tartaric acid, malonic acid, succinic acid, and fumaric acid. Exemplified herein are transdermal accelerants wherein said weak organic acid is citric acid or acetic acid or a combination of both citric acid and acetic acid.

### 2. Fatty Acid Microemulsions

Transdermal delivery formulations for epicutaneous administration of a drug, nutrient, or other compound to a human subject or domestic, veterinary, or agricultural animal comprise: a homogenous mixture of (a) a transdermal accelerant comprising a weak organic acid (as described herein above) and (b) a microemulsion comprising a nonionic emulsifier, water, and an unsaturated long-chain fatty acid.

The fatty acid microemulsions according to the present disclosure are based upon the observation that certain unsaturated fatty acids, particularly, those having a C-12-C26 carbon chain or a C14-C26 carbon chain or a C16-C26 carbon chain when used in combination with a nonionic emulsifier, particularly, those selected from the group consisting of lecithin, carboxylmethylcellulose, a sorbitan ester, and a polysorbate greatly enhance skin permeability and delivery of drugs, nutrients, and other compounds through the skin and into the bloodstream.

As used herein, the terms “non-ionic surfactant,” “non-ionic emulsifier,” and non-ionic detergent” refer collectively to compounds that stabilizes an emulsion by reducing the oil-water interface tension. Non-ionic surfactants, emulsifiers, and detergents are typically amphiphilic compounds having both a polar, hydrophilic, and water-soluble portion and a non-polar, hydrophobic, and lipophilic portion. Non-ionic surfactants, emulsifiers, and detergents employed in the presently disclosed transdermal delivery formulations include lecithin, carboxylmethylcellulose, sorbitan esters, and polysorbates, including polyoxyethylene (20) sorbitan monolaurate (Polysorbate 20), polyoxyethylene (20) sorbitan monopalmitate (Polysorbate 40), polyoxyethylene (20) sorbitan monostearate (Polysorbate 60), and polyoxyethylene (20) sorbitan monooleate (Polysorbate 80).

Polysorbate 80, otherwise known as Tween, is a yellow/golden-colored viscous liquid used as an emulsifier or surfactant in foods, medicines, skincare products, and vaccines. Primarily used to solubilize proteins and is widely used in injectable medications, vaccines.

Polysorbate 80 NF (polyoxyethylene sorbitan monooleate) is derived from RSPO palm oil. It is a non-toxic, nonionic surfactant/emulsifier and a water-soluble yellowish liquid used as a dispersing agent which allows oil and water to mix without the use of alcohol. Polysorbate 80 is a complex mixture consisting of a series of esters and etherates synthesized separately by oleic acid and ethylene oxide with a two-core matrix of sorbitan (Fatty acids in olive oil that are combined with sorbitol. It is plant-derived). Polysorbate 80 helps solubilize ingredients and is considered safe by the FDA for use vitamin and vitamin-mineral preparations which can contain up to 475 milligrams per daily serving of polysorbate 80 (FDA Food Additive Status List). See, CFR 21 (3), $172.840.

Polysorbate 80 has been shown to inhibit reflex pumps involved in the blood-brain barrier (BBB) (e.g., Polysorbate-80 modified neurotoxin microparticles can transport across the BBB). See, Olivier (1999) and Kreuter (2001). Polysorbate 80 can promote TMPP distribution in the brain by increasing drug systemic absorption and then enhanced passive transport of TMPP through the BBB, with the nose-to-brain direct transport percentage decreased to some extent.

The geometric differences between the various types of unsaturated fatty acids, as well as between saturated and unsaturated fatty acids, play an important role in biological processes, and in the construction of biological structures (such as cell membranes). Suitable fatty acids for use in the transdermal delivery formulations disclosed herein include unsaturated long-chain fatty acids comprising a chain of from 16 to 26 carbons and one or more double bonds in a cis configuration. As depicted in Table 3, unsaturated long-chain fatty acids comprising a single double bond in a cis configuration (e.g., Oleic Acid) adopt a “kink” conformation while unsaturated long-chain fatty acids comprising two double bonds in a cis configuration (e.g., Linoleic Acid) adopt a “bend” conformation, and unsaturated long-chain fatty acids comprising three double bonds in a cis configuration (e.g., Linolenic Acid) adopt a “hook” conformation. The effect of this is that, in restricted environments, such as when fatty acids are part of a phospholipid in a lipid bilayer or triglycerides in lipid droplets, cis unsaturated fatty acids increase cellular membrane fluidity by limiting the ability of fatty acids to be closely packed, and thereby reduce the melting temperature of the membrane or of the fat.

In contrast, because the adjacent two hydrogen atoms flanking trans double bond lie on opposite sides of the chain, trans unsaturated long-chain fatty acids comprising a chain of from 16 to 26 carbons and one or more double bonds in a trans configuration exhibit little structural alteration as compared to the corresponding fully-saturated long-chain fatty acid. The effect of this is that, in restricted environments, such as when fatty acids are part of a phospholipid in a lipid bilayer or triglycerides in lipid droplets, trans unsaturated fatty acids decrease cellular membrane fluidity by enhancing the ability of fatty acids to be closely packed, and thereby increase the melting temperature of the membrane or of the fat. See, e.g., Table 3, Stearic Acid (C18:0 saturated), Elaidic Acid (C18:1 in trans), and Linolelaidic Acid (C18:2 in trans).

Thus, disclosed herein are transdermal delivery formulations comprising fatty acid microemulsions wherein a 16 to 26 carbon unsaturated long-chain fatty acid is selected from the group consisting of Sapienic Acid, Palmitoleic Acid, Margoleic Acid, Cis-Vaccenic Acid, Oleic Acid, Petroselinic Acid, Linoleic Acid, Eicosenoic Acid, Gadoleic Acid, Eicosadienoic Acid, Erucic Acid, Docosadienoic Acid, and Nervonic Acid. Exemplified herein are transdermal delivery formulations wherein the 16 to 26 carbon unsaturated long-chain fatty acid is Oleic Acid or Linoleic Acid as well as transdermal delivery formulations wherein the 16 to 26 carbon unsaturated long-chain fatty acid comprises a combination Oleic Acid and Linoleic Acid.

TABLE 3
Structural and Physical Properties of Unsaturated Long-chain Fatty Acids

| | | | Double | |
|---|---|---|---|---|
| | Naturally | Carbons: | Bonds | |
| Fatty | Occurring | Double | (Cis or | |
| Acid | Source | Bonds | Trans) | Secondary Structure |
| Palmitic Acid | Olive Oil | C16:0 | Saturated | 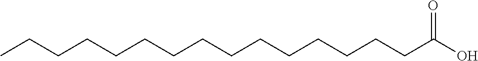 [SMILES: CCCCCCCCCCCCCCCC(=O)O] |
| Sapienic Acid | Human Skin | C16:1 | Cis | 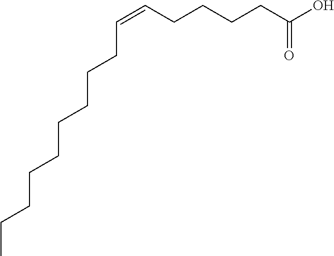 [SMILES: CCCCCCCCC/C=C\CCCCC(=O)O] |
| Palmitoleic Acid | Macadamia Nut Oil Cod Liver Oil Sardine Oil Herring Oil | C16:1 | Cis | 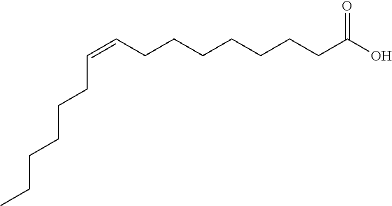 [SMILES: CCCCCC/C=C\CCCCCCCC(=O)O] |
| Trans- Palmitoleic Acid | | C16:1 | Trans | 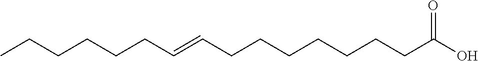 [SMILES: CCCCCC/C=C/CCCCCCCC(=O)O] |
| Margaric Acid | | C17:0 | Saturated | 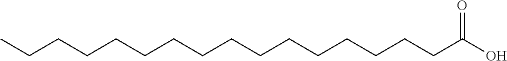 [SMILES: CCCCCCCCCCCCCCCCC(=O)O] |
| Margoleic Acid | Olive Oil | C17:1 | Cis | 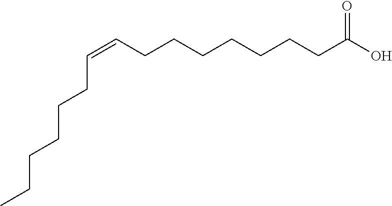 [SMILES: CCCCCC/C=C\CCCCCCCC(=O)O] |
| Stearic Acid | Olive Oil | C18:0 | Saturated | 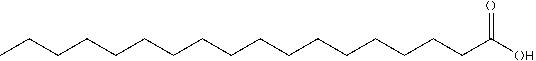 [SMILES: CCCCCCCCCCCCCCCCCC(=O)O] |
| Cis- Vaccenic Acid | | C18:1 | Cis | 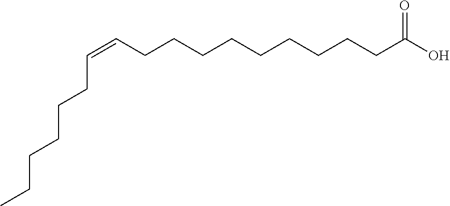 [SMILES: CCCCCC/C=C\CCCCCCCCCC(=O)O] |
| Oleic Acid | Macadamia Nut Oil Olive Oil Animal Fats | C18:1 | Cis Omega-9 Fatty Acid | 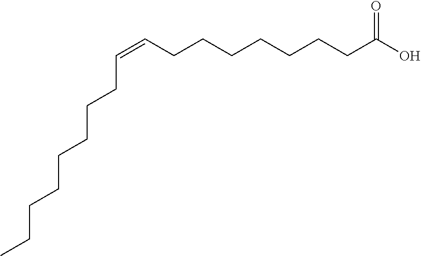 [SMILES: CCCCCCCC/C=C\CCCCCCCC(=O)O] |
| Elaidic Acid | | C18:1 | Trans | 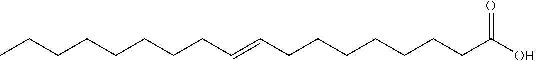 [SMILES: CCCCCCCC/C=C/CCCCCCCC(=O)O] |
| Petroselinic Acid | | C18:1 | Cis | 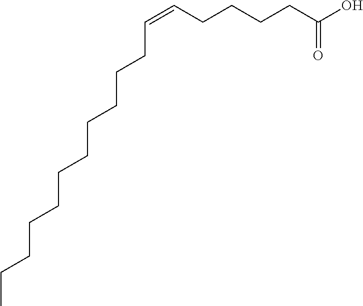 [SMILES: CCCCCCCCCCC/C=C\CCCCC(=O)O] |
| Trans-Vaccenic Acid | Tallow Mutton Butter | C18:1 | Trans | 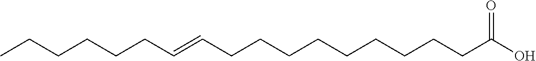 [SMILES: CCCCCC/C=C/CCCCCCCCCC(=O)O] |
| Linoleic Acid | Peanut Oil Olive Oil Maracuja (Passion Fruit) Oil | C18:2 | Cis Essential Omega-6 Fatty Acid | 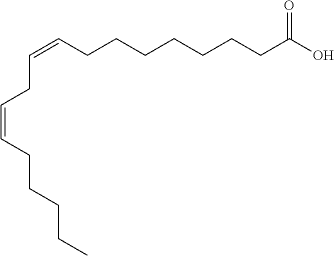 [SMILES: CCCCC/C=C\C/C=C\CCCCCCCC(=O)O] |
| Linolelaidic Acid | Vegetable Oils | C18:2 | Trans | 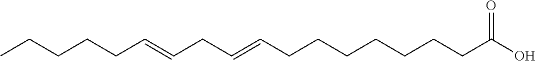 [SMILES: CCCCC/C=C/C/C=C/CCCCCCCC(=O)O] |
| Linelenic Acid | Borage Oil Black Currant Oil Evening Primrose Oil Safflower Oil Flaxseed Oil Chia Seed Oil Walnut Oil | C18:3 | Cis Omega-3 Fatty Acid | 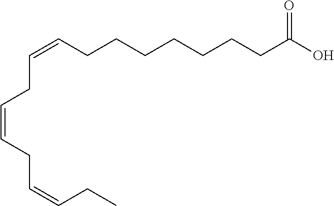 [SMILES: CC/C=C\C/C=C\C/C=C\CCCCCCCC(=O)O] |
| Arachidic Acid | | C20:0 | Saturated | 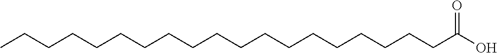 [SMILES: CCCCCCCCCCCCCCCCCCCC(=O)O] |
| Eicosenoic Acid | Various Plant Oils | C20:1 | Cis Omega- 9 Fatty Acid | 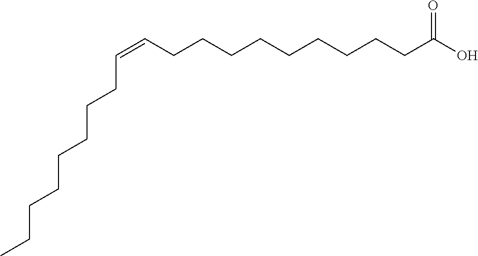 [SMILES: CCCCCCCC/C=C\CCCCCCCCCC(=O)O] |
| Gadoleic Acid | Cod Liver Oil Marine Animal Oils | C20:1 | Cis | 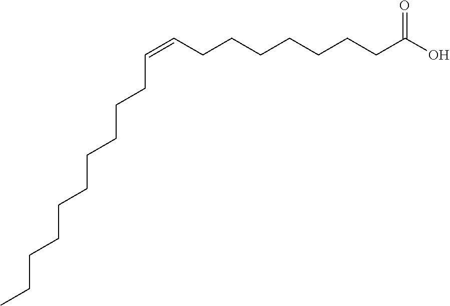 [SMILES: CCCCCCCCCC/C=C\CCCCCCCC(=O)O] |
| Eicosadienoic Acid | | C20:2 | Cis Omega- 6 Fatty Acid | 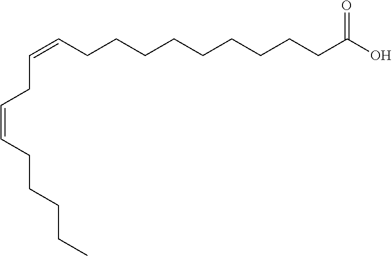 [SMILES: CCCCC/C=C\C/C=C\CCCCCCCCCC(=O)O] |
| Behenic Acid | | C22:0 | Saturated | 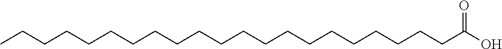 [SMILES: CCCCCCCCCCCCCCCCCCCCCC(=O)O] |
| Erucic Acid | Rapseed Oil Mustard Oil | C22:1 | Cis Omega- 9 Fatty Acid | 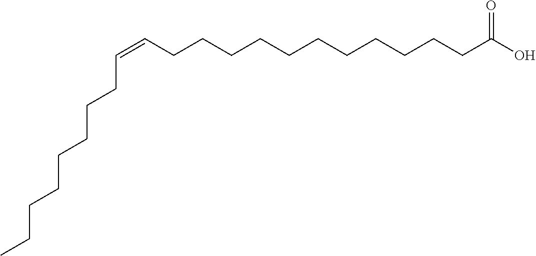 [SMILES: CCCCCCCC/C=C\CCCCCCCCCCCC(=O)O] |
| Docosadienoic Acid | | C22:2 | Cis Omega -6 Fatty Acid | 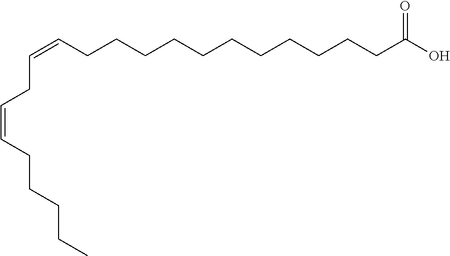 [SMILES: CCCCC/C=C\C/C=C\CCCCCCCCCCCC(=O)O] |
| Brassidic Acid | | C22:1 | Trans | 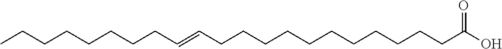 [SMILES: CCCCCCCC/C=C/CCCCCCCCCCCC(=O)O] |
| Lignoceric Acid | | C24:0 | Saturated | 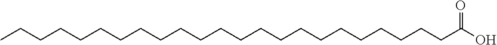 [SMILES: CCCCCCCCCCCCCCCCCCCCCCCC(=O)O] |
| Nervonic Acid | Brain Glycolipids (Nervon) Shingomyelin | C24:1 | Cis Omega- 9 Fatty Acid | 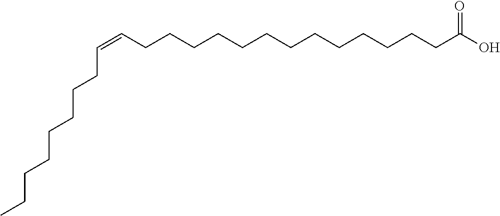 [SMILES: CCCCCCCC/C=C\CCCCCCCCCCCCCC(=O)O] |
| Cerotic Acid | | C26:0 | Saturated | 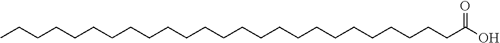 [SMILES: CCCCCCCCCCCCCCCCCCCCCCCCCC(=O)O] |

In related embodiments, the presently disclosed transdermal delivery formulations for epicutaneous administration of a drug, nutrient, or other compound to a human subject or domestic, veterinary, or agricultural animal comprise: a homogenous mixture of (a) a transdermal accelerant comprising a weak organic acid (as described herein above) and (b) a microemulsion comprising a nonionic emulsifier, water, and plant oil.

Suitable plant oils for use in the plant oil microemulsions disclosed herein include vegetable oils, nut oils, and seed oils comprising from 50%-100%, or from 60%-90%, or from 70%-90%, or from 80%-90% of the total unsaturated fatty acid content as long-chain unsaturated fatty acids comprising one or more cis double bonds. Representative suitable plant oils are selected from the group consisting of Macadamia Oil, Maracuja (Passion Fruit) Oil, Safflower Oil, Sunflower Oil, Olive Oil, Avacado Oil, Canola Oil, Coconut Oil, Corn Oil, Cottonseed Oil, Flaxseed/Linseed Oil, Grape Seed Oil, Hemp Seed Oil, Palm Oil, Peanut Oil, Rice Bran Oil, Sesame Oil, Soybean Oil, Brazil Nut Oil, Almond Oil, Walnut Oil, and Pecan Oil.

Exemplified herein are transdermal delivery formulations comprising one or more plant oil as presented in Table 4 “Plant Oil Unsaturated Fatty Acid Profiles,” which include Macadamia Oil, Maracuja (Passion Fruit) Oil, Safflower Oil, Sunflower Oil, Olive Oil, and Almond Oil. In certain aspects, the transdermal delivery formulation comprises Macadamia Oil or Maracuja (Passion Fruit) Oil. In other aspects, the plant oil comprises a combination of Macadamia Oil and Maracuja (Passion Fruit) Oil.

TABLE 4
Plant Oil Unsaturated Fatty Acid Profiles

| | | | |
|---|---|---|---|
| | Average | Primary Structure | |
| | Fatty Acid | (Carbons:Double | Double Bonds |
| Fatty Acid | Content | Bonds) | (Cis or Trans) |

Macadamia Oil

| | | | |
|---|---|---|---|
| Palmitoleic | 19.5% | C16:1 | Cis |
| Acid | | | Omega-7 Fatty Acid |
| Oleic Acid | 61.0% | C18:1 | Cis |
| | | | Omega-9 Fatty Acid |
| Linoleic Acid | 2.2% | C18:2 | Cis |
| | | | Essential omega-6 |
| | | | Fatty Acid |
| Eicosenoic | 2.8% | C20:1 | Cis |
| | | | Omega-3 and -6 |
| | | | Fatty Acids |
| Total % | 85.5% | | |
| Unsaturated | | | |
| Fatty Acids | | | |

Maracuja (Passion Fruit) Oil

| | | | |
|---|---|---|---|
| Palmitoleic | 0.2% | C16:1 | Cis |
| Acid | | | Omega-7 Fatty Acid |
| Vaccenic Acid | 2.5% | C18:1 | Cis |
| Oleic Acid | 18.9% | C18:1 | Cis |
| | | | Omega-9 Fatty Acid |
| Linoleic Acid | 66.9% | C18:2 | Cis |
| | | | Essential Omega-6 |
| | | | Fatty Acid |
| Linolenic Acid | 0.4% | C18:3 | Cis |
| Eicosenoic | 0.1% | C20:1 | Cis |
| Total % | 89.0% | | |
| Unsaturated | | | |
| Fatty Acids | | | |

Safflower Oil

| | | | |
|---|---|---|---|
| Palmitoleic Acid | 0.1% | C16:1 | Cis |
| | | | Omega-7 Fatty Acid |
| Oleic Acid | 23.7% | C18:1 | Cis |
| | | | Omega-9 Fatty Acid |
| Linoleic Acid | 60.8% | C18:2 | Cis |
| | | | Essential Omega-6 |
| | | | Fatty Acid |
| Linolenic Acid | 0.1% | C18:3 | Cis |
| Total % | 84.7% | | |
| Unsaturated | | | |
| Fatty Acids | | | |

Sunflower Oil

| | | | |
|---|---|---|---|
| Oleic Acid | 34.5% | C18:1 | Cis |
| | | | Omega-9 Fatty Acid |
| Linolenic Acid | 52.4% | C18:3 | Cis |
| Eicosenoic Acid | 0.3% | C20:1 | Cis |
| Total % | 87.2% | | |
| Unsaturated | | | |
| Fatty Acids | | | |

Olive Oil

| | | | |
|---|---|---|---|
| Palmitoleic | 0.2% | C16:1 | Cis |
| Acid | | | Omega-7 Fatty Acid |
| Oleic Acid | 63.7% | C18:1 | Cis |
| | | | Omega-9 Fatty Acid |
| Linoleic Acid | 16.9% | C18:2 | Cis |
| | | | Essential Omega-6 |
| | | | Fatty Acid |
| Linolenic Acid | 0.8% | C18:3 | Cis |
| Gadoleic | 0.2% | C20:1 | Cis |
| Total % | 81.8% | | |
| Unsaturated | | | |
| Fatty Acids | | | |

Almond Oil

| | | | |
|---|---|---|---|
| Palmitoleic | 0.3% | C16:1 | Cis |
| Acid | | | Omega-7 Fatty Acid |
| Oleic Acid | 48.6% | C18:1 | Cis |
| | | | Omega-9 Fatty Acid |
| Linoleic Acid | 12.1% | C18:2 | Cis |
| | | | Essential Omega-6 |
| | | | Fatty Acid |
| Total % | 61.0% | | |
| Unsaturated | | | |
| Fatty Acids | | | |

Transdermal delivery formulations may further comprise one or more drug, nutrient, and/or other compound. Exemplified herein are transdermal delivery formulations that further comprise a nitrate source, such as a plant-based nitrate source including, for example, a plant-based nitrate source is selected from the group consisting of arugula, spinach, and beetroot. Exemplified herein are transdermal delivery formulations wherein the plant-based nitrate source is beetroot.

Transdermal delivery formulations may alternatively or additionally comprise one or more (a) viscosity enhancer, (b) nutrient, (c) plant powder or extract, (d) amino acid, and/or (e) vitamin. Representative viscosity enhancers may be selected from the group consisting of lecithin, aloe vera, glycerin, plant oil, animal oil, and collagen.

Representative nutrients may be selected from the group consisting of acetyl-L-carnitine, alpha lipoic acid, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide.

Representative plant powders or extracts may be selected from the group consisting of bacopa powder, bamboo extract powder, beet powder, blueberry extract, ginko biloba, ginger, grape seedextract, green tea, jojoba, nutmeg, olive leaf, pomegranate, and turmeric.

Representative amino acids may be selected from the group consisting of alanine, arginine, leucine, isoleucine, valine, glutamine, glycine, histidine, leucine, lysine, methionine, proline, serine, threonine, and valine.

Within other embodiments, the presently disclosed transdermal delivery formulations comprise a transdermal accelerant wherein the nitrate source is a plant-based nitrate source, such as an arugula, spinach, or beetroot nitrate source.

Within related aspects, the presently disclosed transdermal delivery formulations comprise a carrier serum that comprises a nitrate source, such as a plant-based nitrate source, such as an arugula, spinach, or beetroot nitrate source.

As used herein, the terms “nitrate” and “NO₃” refer interchangeably to an inorganic precursor of “nitric oxide.” Laue, Ullmann's Encyclopedia of Industrial Chemistry (Ed. Weinheim, Wiley-VCH 2006). Plant sources that are especially high in inorganic nitrate include leafy green vegetables, such as spinach and arugula, and beetroot. See, Hord, AJCN 90(1):1 (2009). Dietary nitrate supplementation has been shown to increase endurance exercise performance. McMahon, Sports Medicine 47(4):735 (2017).

Within yet other aspects, the transdermal delivery formulation further comprises (a) a viscosity enhancer, (b) a nutrient, (c) a plant powder or extract, (d) an amino acid, and/or (e) a vitamin. As used herein, the term “viscosity” and “thickness” refers interchangeably to the resistance of a fluid, compound, serum, composition, or formulation to deformation at a given rate. “Viscosity enhancer” refers to compounds that are added to increase the “viscosity” and “thickness” of a composition or formulation. “Viscosity” and “thickness” of a fluid, compound, serum, composition, or formulation typically decreases with increasing temperature. “Viscosity” can be measured with a viscometer, a rheometer, a Zahn cup, or a Ford viscosity cup. For example, oil “viscosity” can be determined using a Cannon-Fenske capillary viscometer, after calibration with 60% sucrose solution in a constant temperature bath regulated to +/−0.05° C. The SI unit of viscosity is the newton-second per square meter (N·s/m²), pascal·second (Pa·s), kilogram per meter per second (kg·m⁻¹·s⁻¹), and Poiseuille (PI). The CGS unit is the poise (P, or g·cm⁻¹·s⁻¹=0.1 Pa·s).

Certain transdermal delivery formulations comprise a viscosity enhancer that is selected from the group consisting of lecithin, aloe vera, glycerin, plant oil, animal oil, and collagen. Other transdermal delivery formulations comprise a nutrient that is selected from the group consisting of acetyl-L-carnitine, alpha lipoic acid, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide.

Further transdermal delivery formulations comprise a plant powder or extract that is selected from the group consisting of bacopa powder, bamboo extract powder, beet powder, blueberry extract, ginko biloba, ginger, grape seedextract, green tea, jojoba, nutmeg, olive leaf, pomegranate, and turmeric. Other transdermal delivery formulations comprise an amino acid that is selected from the group consisting of alanine, arginine, leucine, isoleucine, valine, glutamine, glycine, histidine, leucine, lysine, methionine, proline, serine, threonine, and valine. Yet other transdermal delivery formulations comprise a vitamin that is selected from the group consisting of vitamin A, vitamin B, vitamin B-3, vitamin B-7 & 8 inositol, vitamin B-9 (folic acid), vitamin B-12, vitamin C, vitamin D-3, and vitamin E.

Other embodiments on the present disclosure provides methods for the manufacture of transdermal delivery formulations as disclosed herein for the epicutaneous administrations of drugs, nutrients, and/or other compounds to a human subject or domestic, veterinary, or agricultural animal.

### Exemplary Embodiments

1. A transdermal delivery formulation comprising: a homogenous mixture of (a) a transdermal accelerant comprising a weak organic acid and (b) a microemulsion comprising a nonionic emulsifier, water, and a cis-unsaturated long-chain fatty acid.

2. The transdermal delivery formulation of embodiment 1 wherein said weak organic acid has a median pKa greater than 2.0.

3. The transdermal delivery formulation of any of embodiments 1-2 wherein said weak organic acid has a median pKa of from 3.0 to 5.5.

4. The transdermal delivery formulation of any of embodiments 1-3 wherein said weak organic acid has a median pKa of from 4.0 to 5.0.

5. The transdermal delivery formulation of any of embodiments 1˜4 wherein said weak organic acid has a median pKa of about 4.7.

6. The transdermal delivery formulation of any of embodiments 1-5 wherein said weak organic acid is selected from the group consisting of lactic acid, acetic acid, formic acid, citric acid, oxalic acid, uric acid, malic acid, maleic acid, tartaric acid, malonic acid, succinic acid, and fumaric acid.

7. The transdermal delivery formulation of any of embodiments 1-6 wherein said weak organic acid is citric acid or acetic acid.

8. The transdermal delivery formulation of any of embodiments 1-7 wherein said transdermal accelerant comprises citric acid and acetic acid.

9. The transdermal delivery formulation of any of embodiments 1-8 wherein said nonionic emulsifier is selected from the group consisting of lecithin, carboxylmethylcellulose, a sorbitan ester, and a polysorbate.

10. The transdermal delivery formulation of any of embodiments 1-9 wherein said nonionic emulsifier is a sorbitan ester selected from the group consisting of sorbitan monolaurate, sorbitan monostearate, sorbitan tristearate, and sorbitan monooleate.

11. The transdermal delivery formulation of any of embodiments 1-10 wherein said nonionic emulsifier is a polysorbate selected from the group consisting of polyoxyethylene (20) sorbitan monolaurate (Polysorbate 20), polyoxyethylene (20) sorbitan monopalmitate (Polysorbate 40), polyoxyethylene (20) sorbitan monostearate (Polysorbate 60), and polyoxyethylene (20) sorbitan monooleate (Polysorbate 80).

12. The transdermal delivery formulation of any of embodiments 1-11 wherein said polysorbate is Polysorbate 80.

13. The transdermal delivery formulation of any of embodiments 1-12 wherein said water is distilled water.

14. The transdermal delivery formulation of any of embodiments 1-13 wherein said cis-unsaturated long-chain fatty acid comprises a chain of from 16 to 26 carbons.

15. The transdermal delivery formulation of any of embodiments 1-14 wherein said 16 to 26 carbon cis-unsaturated long-chain fatty acid comprises one or more double bond at position 4-9, or at position 5-8, or at position 6-7.

16. The transdermal delivery formulation of any of embodiments 1-15 wherein said 16 to 26 carbon unsaturated long-chain fatty acid is selected from the group consisting of Sapienic Acid, Palmitoleic Acid, Margoleic Acid, Cis-Vaccenic Acid, Oleic Acid, Petroselinic Acid, Linoleic Acid, Eicosenoic Acid, Gadoleic Acid, Eicosadienoic Acid, Erucic Acid, Docosadienoic Acid, and Nervonic Acid.

17. The transdermal delivery formulation of any of embodiments 1-16 wherein said 16 to 26 carbon unsaturated long-chain fatty acid is Oleic Acid or Linoleic Acid.

18. The transdermal delivery formulation of any of embodiments 1-17 wherein said 16 to 26 carbon unsaturated long-chain fatty acid comprises Oleic Acid and Linoleic Acid.

19. The transdermal delivery formulation of any of embodiments 1-18 wherein said unsaturated long-chain fatty acid is comprised within a plant oil. 20. The transdermal delivery formulation of any of embodiments 1-19 wherein said plant oil is selected from the group consisting of vegetable oil, nut oil and seed oil.

20. The transdermal delivery formulation of any of embodiments 1-19 wherein said plant oil is selected from the group consisting of Macadamia Oil, Maracuja (Passion Fruit) Oil, Safflower Oil, Sunflower Oil, Olive Oil, Avacado Oil, Canola Oil, Coconut Oil, Corn Oil, Cottonseed Oil, Flaxseed/Linseed Oil, Grape Seed Oil, Hemp Seed Oil, Palm Oil, Peanut Oil, Rice Bran Oil, Sesame Oil, Soybean Oil, Brazil Nut Oil, Almond Oil, Walnut Oil, and Pecan Oil.

21. The transdermal delivery formulation of any of embodiments 1-20 wherein said plant oil is selected from the group consisting of Macadamia Oil, Maracuja (Passion Fruit) Oil, Safflower Oil, Sunflower Oil, Olive Oil, and Almond Oil.

22. The transdermal delivery formulation of any one of embodiments 1-21 wherein said plant oil is Macadamia Oil or Maracuja (Passion Fruit) Oil.

23. The transdermal delivery formulation of any one of embodiments 1-22 wherein said plant oil comprises Macadamia Oil and Maracuja (Passion Fruit) Oil.

24. The transdermal delivery formulation of any one of embodiments 1-23 wherein said microemulsion is selected from the group consisting of a cream, an ointment, a liniment, a paste, a film, and a liquid.

25. The transdermal delivery formulation of any one of embodiments 1-24 wherein said transdermal accelerant comprises a nitrate source.

26. The transdermal delivery formulation of any one of embodiments 1-25 wherein said nitrate source is a plant-based nitrate source.

27. The transdermal delivery formulation of any one of embodiments 1-26 wherein said plant-based nitrate source is selected from the group consisting of arugula, spinach, and beetroot.

28. The transdermal delivery formulation of any one of embodiments 1-27 wherein said plant-based nitrate source is beetroot.

29. The transdermal delivery formulation of any one of embodiments 1-28, further comprising a (a) a viscosity enhancer, (b) a nutrient, (c) a plant powder or extract, (d) an amino acid, and/or a (e) vitamin.

30. The transdermal delivery formulation of any one of embodiments 1-29 wherein said formulation comprises a viscosity enhancer selected from the group consisting of lecithin, aloe vera, glycerin, a plant oil, an animal oil, and collagen.

31. The transdermal delivery formulation of any one of embodiments 1-30 wherein said formulation comprises a nutrient selected from the group consisting of acetyl-L-carnitine, alpha lipoic acid, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide.

32. The transdermal delivery formulation of any one of embodiments 1-31 wherein said formulation comprises a plant powder or extract selected from the group consisting of bacopa powder, bamboo extract powder, beet powder, blueberry extract, ginko biloba, ginger, grape seedextract, green tea, jojoba, nutmeg, olive leaf, pomegranate, and turmeric.

33. The transdermal delivery formulation of any one of embodiments 1-32 wherein said formulation comprises an amino acid selected from the group consisting of alanine, arginine, leucine, isoleucine, valine, glutamine, glycine, histidine, leucine, lysine, methionine, proline, serine, threonine, and valine.

34. The transdermal delivery formulation of any one of embodiments 1-33 wherein said formulation comprises a vitamin selected from the group consisting of vitamin A, vitamin B, vitamin B-3, vitamin B-7 & 8 inositol, vitamin B-9 (folic acid), vitamin B-12, vitamin C, vitamin D-3, and vitamin E.

35. The transdermal delivery formulation of any one of embodiments 1-34 wherein said transdermal formulation has a pH of from 2.0 to 6.0.

36. The transdermal delivery formulation of any one of embodiments 1-35 wherein said transdermal formulation has a pH of from 3.0 to 5.0.

37. The transdermal delivery formulation of any one of embodiments 1-36 wherein said transdermal formulation has a pH of from 3.5 to 4.5.

38. The transdermal delivery formulation of any one of embodiments 1-37 wherein said transdermal formulation has a viscosity at 20° C. of from 500 to 10,000 centipoise (cP) or from 1,000 to 5,000 cP, or from 1,500 to 4,000 cP, or from 2,000 to 3,000 cP.

39. The transdermal delivery formulation of any one of embodiments 1-38 wherein said transdermal formulation has a viscosity of about 2,500 cP.

40. A transdermal delivery formulation comprising: a homogenous mixture of (a) a transdermal accelerant comprising a weak organic acid and (b) a microemulsion comprising a nonionic emulsifier, water, and a plant oil comprising an unsaturated long-chain fatty acid.

41. The transdermal delivery formulation of any one of embodiment 40 wherein said weak organic acid has a median pKa of from 2.0 to 6.0.

42. The transdermal delivery formulation of any one of embodiments 40-41 wherein said weak organic acid has a median pKa of from 3.0 to 5.5.

43. The transdermal delivery formulation of any one of embodiments 40-42 wherein said weak organic acid has a median pKa of from 4.0 to 5.0.

44. The transdermal delivery formulation of any one of embodiments 40-43 wherein said weak organic acid has a median pKa of about 4.6.

45. The transdermal delivery formulation of any one of embodiments 40-44 wherein said weak organic acid is selected from the group consisting of lactic acid, acetic acid, formic acid, citric acid, oxalic acid, uric acid, malic acid, maleic acid, tartaric acid, malonic acid, succinic acid, and fumaric acid.

46. The transdermal delivery formulation of any one of embodiments 40-45 wherein said weak organic acid is citric acid or acetic acid.

47. The transdermal delivery formulation of any one of embodiments 40-46 wherein said transdermal accelerant comprises citric acid and acetic acid.

48. The transdermal delivery formulation of any one of embodiments 40-47 wherein said nonionic emulsifier is selected from the group consisting of lecithin, carboxylmethylcellulose, a sorbitan ester, and a polysorbate.

49. The transdermal delivery formulation of any one of embodiments 40-48 wherein said nonionic emulsifier is a sorbitan ester selected from the group consisting of sorbitan monolaurate, sorbitan monostearate, sorbitan tristearate, and sorbitan monooleate.

50. The transdermal delivery formulation of any one of embodiments 40-49 wherein said nonionic emulsifier is a polysorbate selected from the group consisting of polyoxyethylene (20) sorbitan monolaurate (Polysorbate 20), polyoxyethylene (20) sorbitan monopalmitate (Polysorbate 40), polyoxyethylene (20) sorbitan monostearate (Polysorbate 60), and polyoxyethylene (20) sorbitan monooleate (Polysorbate 80).

51. The transdermal delivery formulation of any one of embodiments 40-50 wherein said polysorbate is Polysorbate 80.

52. The transdermal delivery formulation of any one of embodiments 40-51 wherein said water is distilled water.

53. The transdermal delivery formulation of any one of embodiments 40-52 wherein said unsaturated long-chain fatty acid comprises a chain of from 16 to 26 carbons.

54. The transdermal delivery formulation of any one of embodiments 40-53 wherein said 16 to 26 carbon unsaturated long-chain fatty acid comprises one or more double bond in a cis configuration.

55. The transdermal delivery formulation of any one of embodiments 40-54 wherein said 16 to 26 carbon unsaturated long-chain fatty acid is selected from the group consisting of Sapienic Acid, Palmitoleic Acid, Margoleic Acid, Cis-Vaccenic Acid, Oleic Acid, Petroselinic Acid, Linoleic Acid, Eicosenoic Acid, Gadoleic Acid, Eicosadienoic Acid, Erucic Acid, Docosadienoic Acid, and Nervonic Acid.

56. The transdermal delivery formulation of any one of embodiments 40-55 wherein said 16 to 26 carbon unsaturated long-chain fatty acid is Oleic Acid or Linoleic Acid.

57. The transdermal delivery formulation of any one of embodiments 40-56 wherein said 16 to 26 carbon unsaturated long-chain fatty acid comprises Oleic Acid and Linoleic Acid.

58. The transdermal delivery formulation of any one of embodiments 40-57 wherein said plant oil is selected from the group consisting of vegetable oil, nut oil and seed oil.

59. The transdermal delivery formulation of any one of embodiments 40-58 wherein said plant oil is selected from the group consisting of Macadamia Oil, Maracuja (Passion Fruit) Oil, Safflower Oil, Sunflower Oil, Olive Oil, Avacado Oil, Canola Oil, Coconut Oil, Corn Oil, Cottonseed Oil, Flaxseed/Linseed Oil, Grape Seed Oil, Hemp Seed Oil, Palm Oil, Peanut Oil, Rice Bran Oil, Sesame Oil, Soybean Oil, Brazil Nut Oil, Almond Oil, Walnut Oil, and Pecan Oil.

60. The transdermal delivery formulation of any one of embodiments 40-59 wherein said plant oil is selected from the group consisting of Macadamia Oil, Maracuja (Passion Fruit) Oil, Safflower Oil, Sunflower Oil, Olive Oil, and Almond Oil.

61. The transdermal delivery formulation of any one of embodiments 40-60 wherein said plant oil is Macadamia Oil or Maracuja (Passion Fruit) Oil.

62. The transdermal delivery formulation of any one of embodiments 40-61 wherein said plant oil comprises Macadamia Oil and Maracuja (Passion Fruit) Oil.

63. The transdermal delivery formulation of any one of embodiments 40-62 wherein said microemulsion is selected from the group consisting of a cream, an ointment, a liniment, a paste, a film, and a liquid.

64. The transdermal delivery formulation of any one of embodiments 40-63 wherein said transdermal accelerant comprises a nitrate source.

65. The transdermal delivery formulation of any one of embodiments 40-64 wherein said nitrate source is a plant-based nitrate source.

66. The transdermal delivery formulation of any one of embodiments 40-65 wherein said plant-based nitrate source is selected from the group consisting of arugula, spinach, and beetroot.

67. The transdermal delivery formulation of any one of embodiments 40-66 wherein said plant-based nitrate source is beetroot.

68. The transdermal delivery formulation of any one of embodiments 40-67, further comprising a (a) a viscosity enhancer, (b) a nutrient, (c) a plant powder or extract, (d) an amino acid, and/or a (e) vitamin.

69. The transdermal delivery formulation of any one of embodiments 40-68 wherein said formulation comprises a viscosity enhancer selected from the group consisting of lecithin, aloe vera, glycerin, a plant oil, an animal oil, and collagen.

70. The transdermal delivery formulation of any one of embodiments 40-69 wherein said formulation comprises a nutrient selected from the group consisting of acetyl-L-carnitine, alpha lipoic acid, potassium, NALT-Acetyl Tyrosine, NAC, PEA, resveratrol, taurine, palmitate, calcium carbonate, choline bitartrate B-4, creatine, resveratrol, citrulline malate, taurine, magnesium glycinate, carnitine, CoQ10, humic, hyaluronic acid, magnesium, selenium, and zinc oxide.

71. The transdermal delivery formulation of any one of embodiments 40-70 wherein said formulation comprises a plant powder or extract selected from the group consisting of bacopa powder, bamboo extract powder, beet powder, blueberry extract, ginko biloba, ginger, grape seedextract, green tea, jojoba, nutmeg, olive leaf, pomegranate, and turmeric.

72. The transdermal delivery formulation of any one of embodiments 40-71 wherein said formulation comprises an amino acid selected from the group consisting of alanine, arginine, leucine, isoleucine, valine, glutamine, glycine, histidine, leucine, lysine, methionine, proline, serine, threonine, and valine.

73. The transdermal delivery formulation of any one of embodiments 40-72 wherein said formulation comprises a vitamin selected from the group consisting of vitamin A, vitamin B, vitamin B-3, vitamin B-7 & 8 inositol, vitamin B-9 (folic acid), vitamin B-12, vitamin C, vitamin D-3, and vitamin E.

74. The transdermal delivery formulation of any one of embodiments 40-73 wherein said transdermal formulation has a pH of from 2.0 to 6.0.

75. The transdermal delivery formulation of any one of embodiments 40-74 wherein said transdermal formulation has a pH of from 3.0 to 5.0.

76. The transdermal delivery formulation of any one of embodiments 40-75 wherein said transdermal formulation has a pH of from 3.5 to 4.5.

77. The transdermal delivery formulation of any one of embodiments 40-76 wherein said transdermal formulation has a viscosity at 20° C. of from 500 to 10,000 centipoise (cP) or from 1,000 to 5,000 cP, or from 1,500 to 4,000 cP, or from 2,000 to 3,000 cP.

78. The transdermal delivery formulation of any one of embodiments 40-77 wherein said transdermal formulation has a viscosity at 20° C. of about 2,500 cP.

79. The transdermal delivery formulation of any of embodiments 1-78 for epicutaneous administration of a drug, nutrient, or other compound to a human subject or domestic, veterinary, or agricultural animal.

While various embodiments have been disclosed herein, other embodiments will be apparent to those skilled in the art. The various embodiments disclosed herein are for purposes of illustration and are not intended to be limiting, with the true scope and spirit being indicated by the claims. The present disclosure is further described with reference to the following examples, which are provided to illustrate certain embodiments and are not intended to limit the scope of the present disclosure or the subject matter claimed.

### EXAMPLES

### Example 1

### In Vitro Models for Testing Transdermal Delivery Formulations

### (Prophetic)

This Example provides in vitro model systems that may be adapted and employed for the testing various aspects of the transdermal delivery formulations disclosed herein.

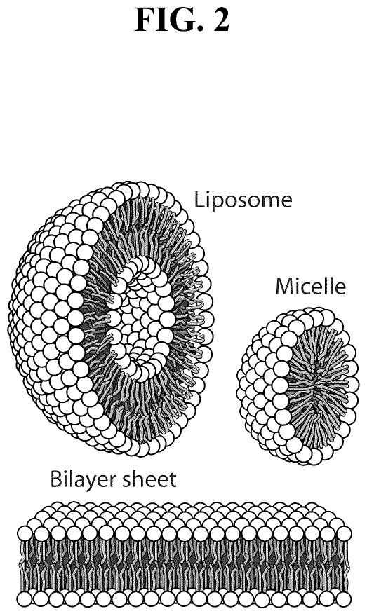

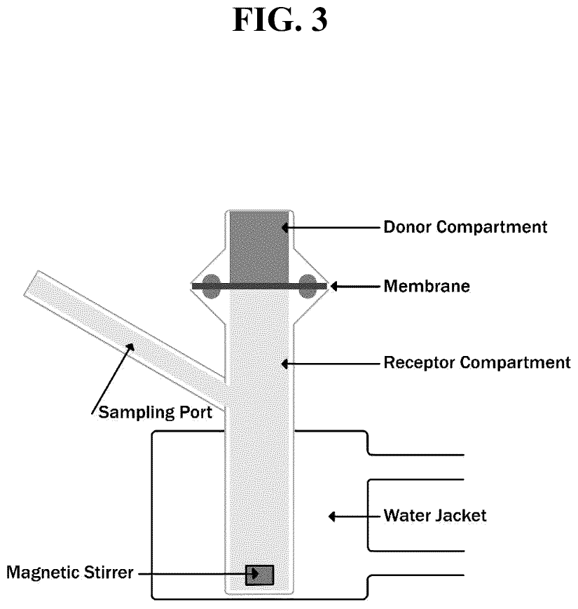

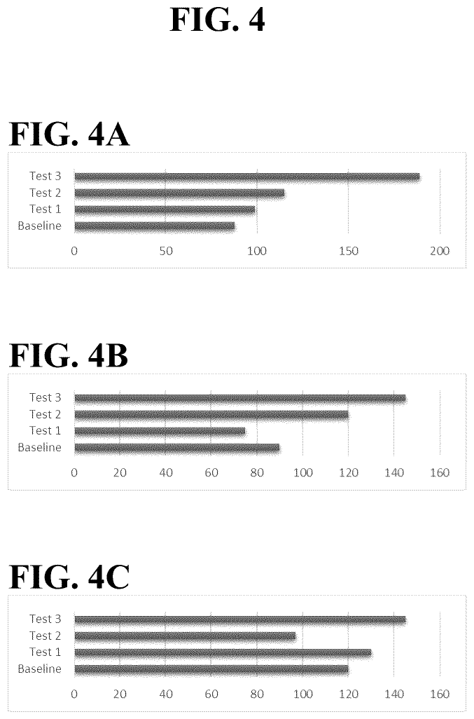

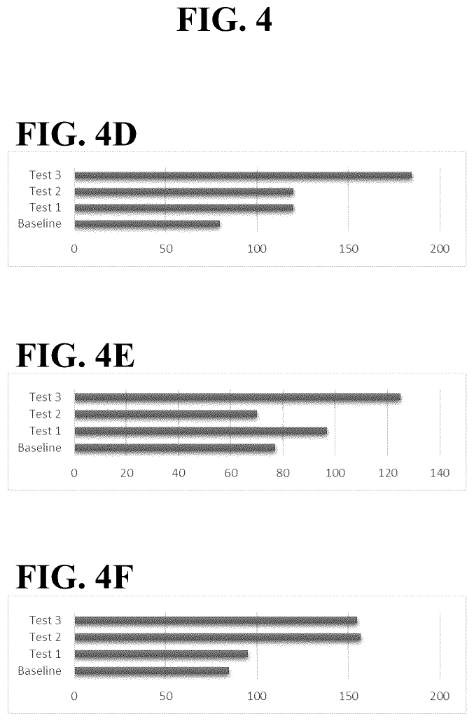

In vitro methods are designed to measure the penetration of compounds, including drugs and nutrients, into the skin and permeation through the stratum corneum and epidermis to the site of vascularization. The Franz diffusion cell (see, FIG. 2) can utilize non-viable skin to measure penetration and permeation only or fresh, metabolically active skin to simultaneously measure permeation and skin metabolism. From, Bartosova, Current Medicinal Chemistry 19:4671 (2012).

Such experiments offer a number of advantages over whole-animal or human volunteer experiments, including saving in time and costs, better reproducibility of results, and less restricted parameter variations. Additional advantages of the in vitro method over the in vivo method are that it can be used equally well with skin from humans and other species, several replicate measurements can be made from the same or number of different subjects, non-radio-labeled test substances which are extensively metabolized can be studied.

In vitro testing is carried out in accordance with “OECD Guideline for the Testing of Chemicals. Draft New Guideline 428: Skin Absorption in vitro method” and Scientific Committee on Consumer Products (SCCP) guidelines.

The in vitro measurement of skin penetration of transdermal delivery formulations includes the application of a test substance in an appropriate formulation (may be radiolabeled) to the surface of a skin sample, which is mounted as a barrier between the donor compartment and the receptor compartment of a diffusion cell. FIG. 2.

The majority of skin absorption studies are conducted using horizontal cells, with the skin surface open to the air. The use of vertical (or side-by-side) cells is more common when evaluating drug delivery systems, such as sonophoresis, iontophoresis or electroporation and requires immersion of both surfaces of the skin preparation, which may result in excessive hydration and possibly skin damage.

Diffusion cells include an inert non-adsorbing material with receptor chamber volumes of about 0.5-10 ml and surface areas of exposed membranes of about 0.2-2 cm². Testing is performed with an appropriate number (i.e. minimum six) skin samples.

The receptor fluid, which must have an adequate capacity to solubilize the test substance, is maintained in contact with underside of the skin from the time of application of a transdermal delivery formulation until the end of the collection of the receptor fluid. Temperature control of the receptor fluid is maintained and monitored throughout the testing. The skin surface temperature in the diffusion cell should be kept at the in vivo skin temperature of 32±1° C. The receptor fluid in static cells is well-stirred throughout the study.

### Example 2

Observational Study with Metabolic Transdermal Delivery Formulation

### (Working)

This Example provides evidence for the therapeutic benefits of a metabolic transdermal delivery formulation through in vivo data presented in Tables 5 and 6 that demonstrate improvements in three key indicia of human health: weight, heart rate, and blood pressure following administration of a transdermal delivery formulation as disclosed herein. Five (5) participants applied to their umbilicus (navel area) approximately 0.35 oz of a Metabolic Transdermal Delivery Formulation twice daily for 21 consecutive days. Each participant's weight, heart rate, and blood pressure were measured on days 0, 7, 14, and 21 and Blood Pressure and Heart Rate were measured.

TABLE 5

Blood Pressure

| Timing | Part 1 | Part 2 | Part 3 | Part 4 |
|---|---|---|---|---|
| Day 0 | 175/86 | 107/72 | 126/76 | 145/75 |
| Day 7 | 153/98 | 94/69 | 113/74 | 128/78 |
| Day 14 | 153/87 | 113/79 | 102/66 | 145/79 |
| Day 21 | 153/98 | 117/69 | | 134/74 |

TABLE 6

Heart Rate

| Timing | Part 1 | Part 2 | Part 3 | Part 4 |
|---|---|---|---|---|
| Day 0 | 57 | 68 | 61 | 58 |
| Day 7 | 70 | 72 | 61 | 52 |
| Day 14 | 64 | 73 | 58 | 58 |
| Day 21 | 64 | 61 | | 57 |

### Example 3

Observational Study with a Transdermal Delivery Formulation

### (Working)

This Example provides evidence for the therapeutic benefits of a transdermal delivery formulation as disclosed herein though in vivo data that demonstrates cognitive improvements in five (5) participants from 60 to 79 years of age and with no previous diagnosis of dementia who applied to their umbilicus (navel area) approximately 0.35 oz of a Focus™ Transdermal Delivery Formulation (or Placebo Control) twice daily for 21 consecutive days.

Improved cognitive function and performance were demonstrated through studies performed by BrainCheck. Inc. (City, State) and Brain Health Restoration (City, State). Four protocols are used during the study: (1) TNS Studies' FOCUS Serum, (2) BrainCheck's online cognitive assessment, (3) EKG and EEG baseline and post-Study, and (4) Brain Health Restoration's MeRT stimulation.

Baseline testing is captured using BrainCheck's online cognitive assessment tool, EKG and EEG for heart and brain activity prior to Study protocol. BrainCheck performs an online cognitive assessment one each participant and provided testing results. Brain Health Restoration provides EEG and EKG and MeRT technology sessions and placebo sessions and EEG and EKG cumulative testing results/statistics pre and post Study.

This Example provides evidence for the therapeutic benefits of a focus transdermal delivery formulation though in vivo data that demonstrates cognitive improvements in five (5) participants who applied to the nape of their neck and shoulders approximately 0.25 oz of a Focus™ Transdermal Delivery Formulation (or Placebo Control) twice daily for 21 consecutive days.

Improved cognitive function and performance were demonstrated through studies performed with the Roberto App (City, State). Testing scores were tracked and indicators of improvement were assessed in the following areas: (1) mental focus, (2) hand/eye coordination, (3) eye tracking, (4) auditory and visual recognition & recollection, and (5) reduction in stress and mental fatigue. 100% of Study Participants reported improved or less stress during the Study. 83% of Study Participants' sleep was improved. 66% of Study Participants reported feeling calmer while on the Serum.

The results of this study are presented in Table 7. The average change in Total Score from baseline to Test 3, as reported by BrainCheck, was 10%. Improvements in executive function were demonstrated by a 27% improvement from baseline for Stroop Color Interference measurements of the ability of a human subject or domestic, veterinary, or agricultural animal to inhibit reactions and control impulses.

Human subject or domestic, veterinary, or agricultural animals in the 51-60 age range showed the greatest improvement in Total Score, 19.5%. Attention improved by 29%, Mental Flexibility improved by 4% (Ages 71-80), Executive Function (digital symbol substitution) improved by 15.5% (Ages 41-50), Executive Function (stroop) improved by 44% (Ages 41-50), Immediate Memory improved by 16.6% (Ages 51-60), and Delayed Memory improved by 27% (Ages 71-80).

TABLE 7
ATTENTION
Trails A

| | |
|---|---|
| Baseline | 88 |
| Test 1 | 99 |
| Test 2 | 115 |
| Test. 3 | 189 |

MENTAL FLEXIBILITY
Trails B

| | |
|---|---|
| Baseline | 90 |
| Test 1 | 75 |
| Test 2 | 120 |
| Test 3 | 145 |

EXECUTIVE FUNCTION
Digital Symbol Substitution

| | |
|---|---|
| Baseline | 120 |
| Test 1 | 130 |
| Test 2 | 97 |
| Test 3 | 185 |

EXECUTIVE FUNCTION
Stroop

| | |
|---|---|
| Baseline | 80 |
| Test 1 | 120 |
| Test 2 | 120 |
| Test 3 | 155 |
| MEMORY | |

Immediate Recognition

| | |
|---|---|
| Baseline | 77 |
| Test 1 | 97 |
| Test 2 | 70 |
| Test 3 | 125 |

MEMORY
Delayed Recognition

| | |
|---|---|
| Baseline | 85 |
| Test 1 | 95 |
| Test 2 | 157 |
| Test 3 | 155 |

COMBINED TEST RESULTS

| | |
|---|---|
| Baseline | 76 |
| Test 1 | 85 |
| Test 2 | 105 |
| Test 3 | 97 |

The scope of the disclosure is indicated by the appended claims rather than by the foregoing description, and all changes that come within meaning and range of equivalency of the claims are intended to be embraced herein.

1. A transdermal delivery formulation comprising: a homogenous mixture of:
(a) a drug or nutrient;
(b) a first composition comprising a transdermal accelerant comprising a weak organic acid having a pKa greater than 2.0, in an amount effective to result in transdermal systemic delivery of the drug or nutrient, wherein the weak organic acid comprises lactic acid, acetic acid, formic acid, citric acid, oxalic acid, uric acid, malic acid, maleic acid, tartaric acid, malonic acid, succinic acid, or fumaric acid; and
(c) a second composition comprising a microemulsion comprising a nonionic emulsifier, water, and a cis-unsaturated long-chain fatty acid,
wherein said transdermal delivery formulation has a pH of from 2.0 to 6.0,
wherein said transdermal delivery formulation has a viscosity at 20° C. of from 1,500 to 4,000 cP, and
wherein the formulation, upon application to a subject, results in transdermal systemic delivery of the drug or nutrient.

2. The transdermal delivery formulation of claim 1, wherein said weak organic acid has a median pKa of from 2.0 to 6.0, from 3.0 to 5.5, from 4.0 to 5.0, or about 4.7.

3. (canceled)

4. The transdermal delivery formulation of claim 1, wherein said weak organic acid is citric acid and/or acetic acid.

5. The transdermal delivery formulation of claim 1, wherein said nonionic emulsifier is selected from the group consisting of lecithin, carboxylmethylcellulose, a sorbitan ester, and a polysorbate.

6. The transdermal delivery formulation of claim 5, wherein said nonionic emulsifier is a sorbitan ester selected from the group consisting of sorbitan monolaurate, sorbitan monostearate, sorbitan tristearate, and sorbitan monooleate or a polysorbate selected from the group consisting of polyoxyethylene (20) sorbitan monolaurate (Polysorbate 20), polyoxyethylene (20) sorbitan monopalmitate (Polysorbate 40), polyoxyethylene (20) sorbitan monostearate (Polysorbate 60), and polyoxyethylene (20) sorbitan monooleate (Polysorbate 80).

7. The transdermal delivery formulation of claim 6, wherein said polysorbate is Polysorbate 80.

8. The transdermal delivery formulation of claim 1, wherein said water is distilled water.

9. The transdermal delivery formulation of claim 1, wherein said cis-unsaturated long-chain fatty acid comprises a chain of from 16 to 26 carbons.

10. (canceled)

11. The transdermal delivery formulation of claim 9, wherein said 16 to 26 carbon cis-unsaturated long-chain fatty acid is selected from the group consisting of Sapienic Acid, Palmitoleic Acid, Margoleic Acid, Cis-Vaccenic Acid, Oleic Acid, Petroselinic Acid, Linoleic Acid, Eicosenoic Acid, Gadoleic Acid, Eicosadienoic Acid, Erucic Acid, Docosadienoic Acid, and Nervonic Acid.

12. The transdermal delivery formulation of claim 11, wherein said 16 to 26 carbon cis-unsaturated long-chain fatty acid is Oleic Acid and/or Linoleic Acid.

13. The transdermal delivery formulation of claim 1, further comprising one or more plant oils selected from the group consisting of Macadamia Oil, Maracuja (Passion Fruit) Oil, Safflower Oil, Sunflower Oil, Olive Oil, Avocado Oil, Canola Oil, Coconut Oil, Corn Oil, Cottonseed Oil, Flaxseed/Linseed Oil, Grape Seed Oil, Hemp Seed Oil, Palm Oil, Peanut Oil, Rice Bran Oil, Sesame Oil, Soybean Oil, Brazil Nut Oil, Almond Oil, Walnut Oil, and Pecan Oil,
wherein the one or more plant oils comprises the cis-unsaturated long-chain fatty acid.

14. The transdermal delivery formulation of claim 13, wherein said one or more plant oils comprise at least one of Macadamia Oil and Maracuja (Passion Fruit) Oil.

15. The transdermal delivery formulation of claim 1, wherein said microemulsion is selected from the group consisting of a cream, an ointment, a liniment, a paste, a film, and a liquid.

16. (canceled)

17. The transdermal delivery formulation of claim 1, wherein the first composition further comprises a nitrate source, wherein said nitrate source is a plant-based nitrate source selected from the group consisting of arugula, spinach, and beetroot.

18. (canceled)

19. (canceled)

20. (canceled)
